(19) United States
(12) Patent Application Publication
(10) Pub. No.: US 2013/0034466 A1
(43) Pub. Date: Feb. 7, 2013

(54) SAMPLE ANALYZER

(71) Applicants: Yuji Wakamiya, Kobe-shi (JP); Tomoyuki Nishida, Ashiya-shi (JP)

(72) Inventors: Yuji Wakamiya, Kobe-shi (JP); Tomoyuki Nishida, Ashiya-shi (JP)

(21) Appl. No.: 13/564,977

(22) Filed: Aug. 2, 2012

(30) Foreign Application Priority Data
Aug. 3, 2011 (JP) JP2011-170498

(51) Int. Cl. G01N 33/53; G01N 33/50

(52) U.S. Cl. 422/64; 422/63

(57) ABSTRACT

A measuring section 2 is provided with a rotatable reaction table 200 for rapidly starting the measurement of a new sample even when an abnormal operation has stopped the measurement operations of many cuvettes. A controller 4 detects a residual cuvette remaining on the reaction table 200 when a measurement start instruction is received. When a residual cuvette is present, the controller 4 determines whether the residual cuvette will cause interference, and when the residual cuvette will not cause interference, sets the new sample on the reaction table 200, rotates the reaction table 200 and sequentially processes the cuvettes. In conjunction therewith, when a residual cuvette is moved to the end position C56, the liquid is aspirated and discarded through a discarding port W via a drain unit 282.

### RELATED APPLICATIONS

This application claims priority under 35 U.S.C. §119 to Japanese Patent Application No. 2011-170498 filed on Aug. 3, 2011, the entire content of which is hereby incorporated by reference.

### BACKGROUND OF THE INVENTION

1. Field of the Invention

The present invention relates to a sample analyzer for measuring a sample contained in a cuvette.

2. Description of the Related Art

There are conventional sample analyzers for dispensing a sample and reagents into a cuvette to be measured (for example, United States Patent Publication No. 2009/215183.)

The sample analyzer disclosed in United States Patent Publication No. 2009/215183 is provided with a primary reaction section with a rotating table part for holding a plurality of cuvettes, a primary BF separation section for holding and performing BF separation on a cuvette received from the primary reaction section, a secondary reaction section with a rotating table for holding a plurality of cuvettes previously subjected to BF separation by the primary BF separation section, a secondary BF separation section for holding and performing BF separation on a cuvette received from the secondary reaction section, and a detection section. In this sample analyzer, a cuvette containing a sample is sequentially moved to the primary reaction section, primary BF separation section, secondary reaction section, secondary BF separation section, and detection section, then the detection section detects the measurement object substance within the cuvette. After detection has been completed, the cuvette is discarded in a disposal bag.

When an abnormality occurs in a reaction section or BF separation section during a measurement operation, the sample analyzer disclosed in United States Patent Publication No. 2009/215183 interrupts the measurement process of the cuvette upstream from the reaction section or BF separation section in which the abnormality occurred, and continues the measurement operation of the cuvette downstream from the reaction section or BF separation section in which the abnormality occurred. When the user issues instruction to restart measurement after recovery from the abnormality, this analyzer discards all cuvettes involved in the interrupted measurement operation, and after all cuvettes have been discarded, restarts the measurement operation with a new sample.

In the analyzer disclosed in United States Patent Publication No. 2009/215183, the measurement operations of several cuvettes within the apparatus are suspended when and abnormality occurs at a location in a final stage process of an ongoing measurement operation, such as the secondary reaction section and secondary BF separation section. In this case, a longer time is required until starting measurement of a new sample because time is consumed while discarding all of the cuvettes in the apparatus.

### SUMMARY OF THE INVENTION

The scope of the present invention is defined solely by the appended claims, and is not affected to any degree by the statements within this summary.

A first aspect of the present invention is a sample analyzer comprising:

a plurality of processing stations for performing processes to obtain a measurement of a sample contained in a cuvette;
a cuvette transporting section configured to sequentially transport a plurality of cuvettes, each cuvette containing a sample, to the plurality of processing stations;
a discarding station for discarding the cuvette transported to a predetermined position by the cuvette transporting section;
an abnormality detecting section for detecting an abnormality at the plurality of processing stations; and
a controller configured for performing operations, comprising:
(a) stopping the processing of a cuvette or cuvettes when an abnormality is detected by the abnormality detecting section;
(b) when an instruction for starting the processing is received after recovery from an abnormality, transporting a cuvette or cuvettes containing a new sample or samples, held by the cuvette transporting section, to the plurality of processing stations and transporting the cuvette or cuvettes for which processing has been stopped to a predetermined position; and
(c) discarding the cuvette or cuvettes transported to the predetermined position by the discarding station.

A second aspect of the present invention is a sample analyzer comprising:

a cuvette transporting section comprising a rotating table for sequentially transporting a plurality of cuvettes, each containing a sample;
a reagent dispensing section for dispensing reagent to react with a measurement object substance contained in the sample in the cuvette transported to a first position by the cuvette transporting section;
a detecting section for performing processing to detect the measurement object substance in the cuvette transported to a second position downstream from the first position by the cuvette transporting section;
an aspiration removal section for aspirating the liquid within a cuvette after the detecting section has performed the measurement object substance detection processing;
a cuvette discarding section for discarding the cuvette from which the liquid has been aspirated;
a cuvette moving section configured to move the cuvette at the detecting section to the aspiration removal section, and move the cuvette from which the liquid has been aspirated to the cuvette discarding section;
a controller for sequentially executing measurement operations on each cuvette hold in the cuvette transporting section by controlling the operations of the reagent dispensing section, aspiration removal section, and cuvette transporting section; and
an abnormality detecting section for detecting an abnormality, wherein
the controller performs operations, comprising:
(a) stopping the measurement operation when an abnormality is detected by the abnormality detecting section during a measurement operation;
(b) when a measurement start instruction is received after the abnormality recovery process, sequentially executing measurement operations on the cuvettes containing new sample and controlling the cuvette moving section to sequentially move the cuvettes left unmeasured due to the stopped operation from the cuvette transporting section to the aspiration removal section, and the cuvette discarding section in conjunction with the measurement operation.

The effect or significance of the present invention will become clear from the following description of the embodiment. Of course the following embodiment is a single example realizing the present invention, and the present invention is in no respect limited to the embodiment below.

### BRIEF DESCRIPTION OF THE DRAWINGS

FIG. 1 is a perspective view showing the general structure of an immunoanalyzer of the present embodiment;

FIG. 2 is a plan view showing the structure when viewing the inside of a measuring unit 2 from above;

FIG. 3 is a flow chart showing the flow of the measurement process performed by the measuring unit 2;

FIG. 4 is a flow chart illustrating the operation flow from when an abnormality occurs in a measurement until measurement is restarted;

FIG. 5 is a flow chart illustrating subroutine of step S208 of FIG. 4;

FIG. 6 is a flow chart illustrating the subroutine of step S210 of FIG. 4;

FIG. 7 is a flow chart illustrating subroutine of step S212 of FIG. 4;

FIG. 8 is a flow chart illustrating subroutine of step S213 of FIG. 4;

FIG. 9A schematically shows the structure of a cuvette arrangement buffer;

FIG. 9B schematically shows the measurable arrangement pattern;

FIG. 9C illustrates the comparison process of the measurable arrangement pattern and the cuvette arrangement buffer;

FIG. 10 schematically shows measurable arrangement pattern against the reaction table 200; and

FIG. 11 is a block diagram briefly showing the structure of the abnormality detecting unit for detecting an abnormality in the measurement process.

### DETAILED DESCRIPTION OF THE PREFERRED EMBODIMENTS

The preferred embodiments of the present invention will be described hereinafter with reference to the drawings.

An embodiment of the present invention applies the present invention to an immunoanalyzer for examining various items such as hepatitis B, hepatitis C, tumor markers, and thyroid hormone using a sample such as blood.

The immunoanalyzer of the embodiment is described below using the drawings.

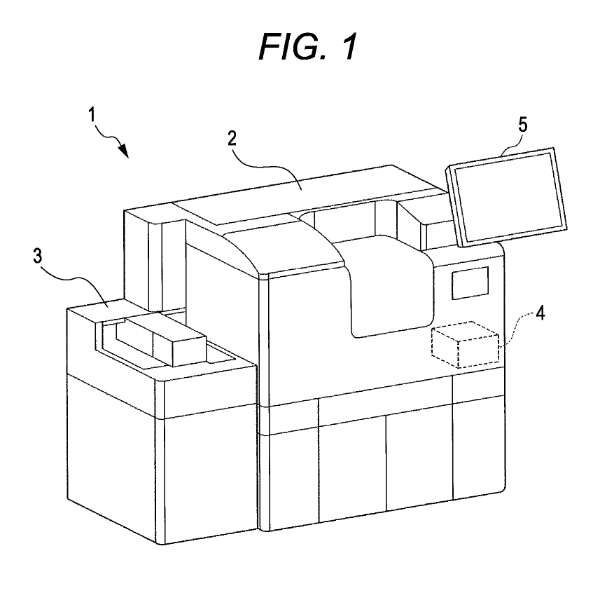

As shown in FIG. 1, the immunoanalyzer 1 is provided with a measuring section 2, sample transporting section 3, controller 4, and display/operation section 5. The sample transporting section 3 transports the sample rack holding sample containers that contain serum sample. The measuring section 2 aspirates and measures the sample from the sample container held in the sample rack transported by the sample transporting section 3. The display/operation section 5 is provided with a touch panel, and has the functions of displaying the measurement results acquired by the measuring section 2, and receiving a measurement start instruction. The controller 4 is provided in the measuring section 2, and controls each section of the immunoanalyzer 1.

### Structure of the Measuring Section

The flow of the measurement operation performed by the measuring section 2 is briefly described. First, the serum sample composing the measurement object is mixed with a buffer solution ((R1 reagent). A reagent (R2 reagent) containing magnetic particles carrying capture antibody for binding, via antigen-antibody reaction, to an antigen (measurement object substance) contained in the sample is added to the obtained liquid mixture. The magnetic particles carrying the capture antibody bound to the antigen are attracted to a magnet (not shown in the drawing) of the primary BF (bound free) separation section, and the unreacted reagent component that is not bound to the capture antibody is separated and removed. A labeled antibody (R3 reagent) is then added to label the antibody bound to the antigen via the antigen-antibody react. The magnetic particles carrying the capture antibody bound to the antigen and labeled antibody are attracted to the magnet of the secondary BF separation section 230, and the unreacted labeled antibody is separated and removed. After adding a luminescent substrate (R5 reagent), which luminesces via reaction between the labeled antibody and a dispersion liquid (R4 reagent), the amount of light produced by the reaction between the labeled antibody and the luminescent substrate is measured. The measurement object antigen contained in the sample bound to the labeled antibody can be quantified through this process.

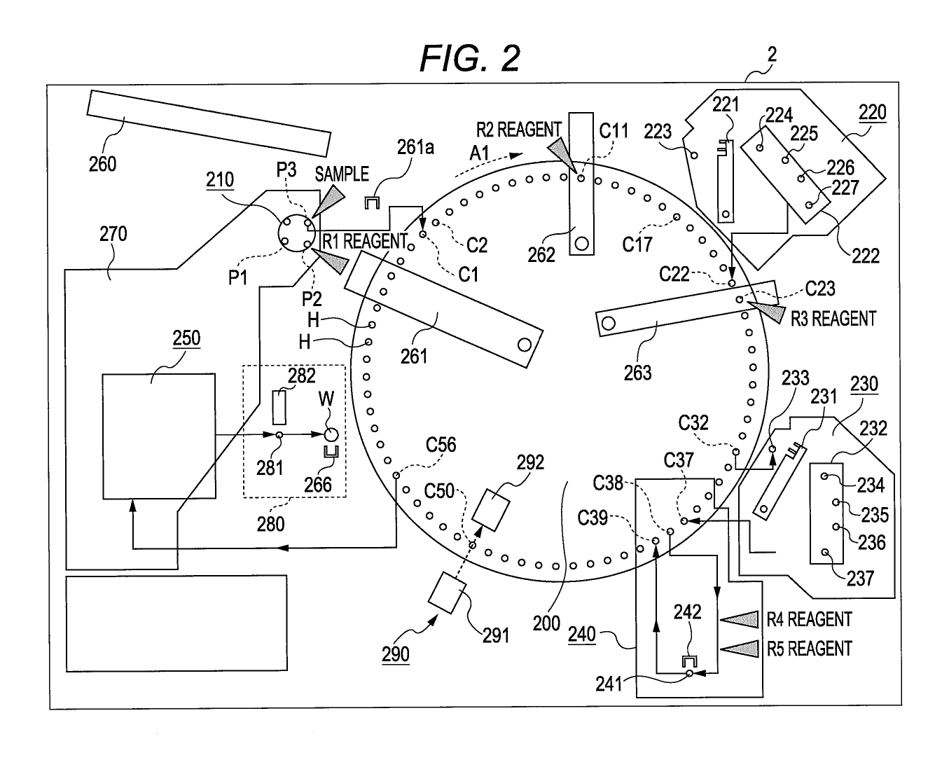

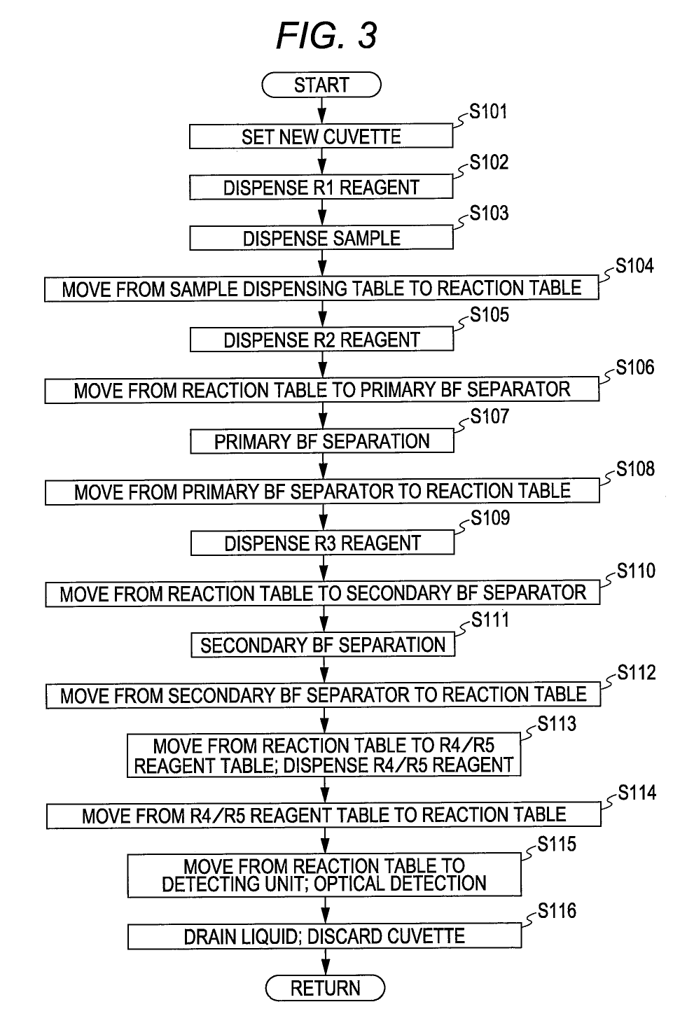

The structure of the measuring section 2 is described in detail below together with the flow of the measurement process performed by the measuring section 2 with reference to FIGS. 2 and 3.

The measuring section 2 incorporates a plurality of processing stations, mainly a reaction table 200, cuvette table 210, cuvette supplying section 270, sample dispensing arm 260, R1 reagent dispensing arm 261, R2 reagent dispensing arm 262, primary BF separation section 220, R3 reagent dispensing arm 263, secondary BF separation section 230, R4/R5 reagent supplying section 240, detecting section 250, and discarding section 280.

The cuvette supplying section 270 houses a plurality of cuvettes. The cuvette supplying section 270 sequentially supplies cuvettes, one at a time, to the cuvette setting position P1 on the cuvette table 210.

The cuvette table 210 is a rotatable table with four cuvette holders. The cuvette table 210 receives the cuvette from the cuvette supplying section 270 at the cuvette setting position P1. The cuvette table 210 rotates to sequentially transport the received cuvette to the R1 reagent dispensing position P2 and the sample dispensing position P3.

The R1 reagent dispensing arm 261 aspirates the R1 reagent from the R1 reagent container disposed at a predetermined position, and dispenses the R1 reagent to the cuvette at the R1 reagent dispensing position P1 (step S 102).

The sample dispensing arm 260 aspirates the sample within the sample container transported by the sample transporting section 3, and dispenses the aspirated sample to the cuvette disposed at the sample dispensing position P3 (step S103).

A catcher 261a is provided near the cuvette table 210. The catcher 261a removes the cuvette, which has received the dispensed sample at the sample dispensing position P3, from the cuvette table 210, and sets the cuvette at the transport start position C1 on the reaction table 200 (step S104).

The reaction table 200 is configured as a rotatable table with cuvette holders H capable of holding a cuvette. There are seventy individual cuvette holders H annularly arranged at fixed spacing along the outer periphery of the reaction table 200. Cuvettes set in the cuvette holders H are heated to approximately 42° C. Hence, the heating promotes reaction of the various reagents and the sample in the cuvette.

The reaction table 200 rotates clockwise (A1 direction) a fixed angle θ at 18 second intervals (1 turn). Hence, the reaction table 200 cycles the cuvettes set in the cuvette holders H from the transport start position C1, sequentially through positions C2, C3 and the like, until the cuvette reaches the transport end position C56 where the cuvette is removed from the table by a catcher 266, which will be described later. The fixed angle θ is the angle required to move the cuvette holder H at an optional position Cn to an adjacent position Cn+1 in the arrow A1 direction, specifically, θ=360/70 degrees).

Hereinafter, C1 is referred to as the transport start position, and C56 is referred to as the transport end position. Counting from the transport start position C1 in a clockwise direction, the nth position is referred to as Cn. Only locations from C1 through C70 necessary to the description are labeled in FIG. 2. In FIG. 2, the structure of the measuring section 2 is delineated by solid lines, and the symbols representing positions are indicated by dashed lines. In FIG. 3, the transport performed by the reaction table 200 is abbreviated, although the reaction table 200 is shown moving the cuvette continuously from step S104 through S115.

The R2 reagent dispensing arm 262 aspirates the R2 reagent from the R2 reagent container set at a predetermined position, and dispenses the aspirated R2 reagent to the cuvette at the R2 reagent dispensing position C11 (step S 105). The cuvette that received the dispensed R2 reagent is transported to the position C17 by rotating the reaction table 200.

The primary BF separation section 220 has a catcher 221 and a primary BF table 222. The cuvette, which received the dispensed sample, R1 reagent and R2 reagent and was moved to the position C17 on the reaction table 200, is removed from the reaction table 200 by the catcher 221, and the catcher 221 then moves the cuvette to the standby section 223 of the primary BF separation section 220 (step S106).

The primary BF table 222 has four holders 224 through 227. The catcher 221 removes the cuvette disposed at the standby section 223, and sets the cuvette in any one of the holders 224 through 227.

The primary BF separation section 220 locally collects the magnetic particles within the cuvettes as the cuvettes set in the holders 224 through 227 approach the magnet, and removes the components that have not bonded to the capture antibody from the sample within the cuvette. Then, the primary BF separation section 220 dispenses and mixes wash solution into the cuvette, and again collects the magnetic particles, and removes the wash solution. This process is repeated to remove the unreacted reagent component from the cuvette (step S107).

When the primary BF separation process is completed, the catcher 221 removes the cuvette and returns the cuvette to position C22 on the reaction table 200 (step S108). The cuvette subjected to the completed primary BF separation is transported from position C22 to position C23 by the rotation of the reaction table 200.

The R3 reagent dispensing arm 263 aspirates the R3 reagent from the R3 reagent container set at a predetermined position, and dispenses the aspirated R3 reagent to the cuvette at the R3 reagent dispensing position C23 on the reaction table 200 (step S109). The cuvette that received the dispensed R3 reagent is transported from the position C23 to the position C32 by rotating the reaction table 200.

The secondary BF separation section 230 has a catcher 231 and a secondary BF table 232. The cuvette, which received the dispensed R3 reagent and was moved to the position C32 on the reaction table 200, is removed from the reaction table 200 by the catcher 231, and the catcher 231 then moves the cuvette to the standby section 233 of the secondary BF separation section 230 (step S110).

The secondary BF table 232 has four holders 234 through 237. The catcher 231 removes the cuvette disposed at the standby section 233, and sets the cuvette in any one of the holders 234 through 237.

The secondary BF separation section 230 locally collects the magnetic particles within the cuvettes as the cuvettes set in the holders 234 through 237 approach the magnet, and removes the R3 reagent that has not reacted with the sample within the cuvette. Then, the secondary BF separation section 230 dispenses and mixes wash solution into the cuvette, and again collects the magnetic particles, and removes the wash solution. This process is repeated to remove the unreacted reagent component from the cuvette (step S111).

When the secondary BF separation process is completed, the catcher 231 removes the cuvette and returns the cuvette to position C37 on the reaction table 200 (step S112). The cuvette subjected to the completed secondary BF separation is transported from position C37 to position C38 by the rotation of the reaction table 200.

The R4/R5 reagent supplying section 240 has a holder 241 for holding a cuvette, and a catcher 242.

The catcher 242 removes the cuvette at the position C38 on the reaction table 200, and places the cuvette in the holder 241. The R4/R5 reagent supplying section 240 dispenses the R4 reagent and R5 reagent to the cuvette held by the catcher 242 in the middle of the transport pass toward the holder 241. The catcher 242 sets the cuvette containing the dispensed R4 and R5 reagents in the holder 241 (step S113).

The catcher 242 removes the cuvette set in the holder 241, and moves the cuvette toward the position C30 on the reaction table 200 and returns to the position C39 of the reaction table 200 (step S114). The cuvette containing the dispensed R4 and R5 reagents is then moved from the position C39 to the transport end position C56 via the rotation of the reaction table 200.

The detecting section 250 has a darkroom capable of accommodating the cuvette, and this darkroom has the function of measuring the light emitted from the cuvette in the darkroom.

The discarding section 280 has a catcher 266, drain holding section 281, drain section 282, and discarding port W.

The catcher 266 has the function of holding and moving the cuvette. The drain section 282 has the functions of aspirating the liquid from the cuvette held by the drain holding section 281, and discharging the liquid to outside the apparatus. The drain port W is configured by a hole that communicates with a discarding bag disposed below the apparatus.

When the cuvette is moved to the transport end position C56 of the reaction table 200, the catcher 266 removes the cuvette and moves the cuvette to the detecting section 250. The detecting section 250 measures the amount of antigen contained in the sample by acquiring the light produced by the reaction process between the luminescent substrate and labeled antibody in the cuvette using a photomultiplier tube (step S115).

When detection is completed by the detecting section 250, the catcher 266 removes the cuvette from the detecting section 250 and moves the cuvette to the drain holding section 281, then the drain section 282 aspirates the liquid from the cuvette and discharges the liquid. The catcher 266 moves the cuvette to above the discarding port W, and the cuvette is discarded from the discarding port W to the discarding bag when the catcher 266 releases the grip on the cuvette (step S116).

The description above pertains to the main structure of the measuring section 2 and the measurement flow series by each part thereof. Note that the measurement flow shown in FIG. 3 shows only the series of measurement flow related to a single cuvette, whereas the series of measurement flow occurs in parallel for a plurality of cuvettes when the measuring section 2 is actually operating.

The measuring section 2 is further provided with a cuvette detector 290 which is disposed near the reaction table 200. The cuvette detector 290 has a light emitting part 291 disposed at the outer side of the cuvette array on the reaction table 200, and a light receiving part 292 disposed at the inner side of the cuvette array on the reaction table 200. The light emitting part 291 emits light on the cuvette at position C50 (hereinafter referred to as the cuvette detection position) on the reaction table 200. The light receiving part 292 is fixedly attached to the inner side of the reaction table 200. The light receiving part 292 receives the light emitted from the light emitting part 291 toward the cuvette. The cuvette detector 290 with this structure detects the presence of the cuvette at the cuvette holder H disposed at the cuvette detection position C50. Specifically, when the light emitting part 291 emits light and a cuvette is present in the cuvette holder H at the cuvette detection position C50, the cuvette impinges the cuvette is and scattered and the amount of light reaching the light receiving part 292 is decreased compared to when a cuvette is not present. Therefore, a presence of a cuvette in the cuvette holder H can be ascertained when the amount of light receives by the light receiving part 292 is equal to or greater than a predetermined value, and the absence of a cuvette in the cuvette holder H can be ascertained when the amount of light is less than the predetermined value.

The cuvette detector 290 is used in the priority cuvette discarding determination process (described later), and not in the series of the measurement flow shown in FIG. 3.

### Measurement Restart Operation

The operation of the immunoanalyzer 1 of the present embodiment is described below from the time an abnormality occurs in a measurement until measurement is restarted.

The immunoanalyzer 1 determines whether an abnormality has occurred in the measurement operation when the series of measurement flow shown in FIG. 3 is performed for a plurality of cuvettes (step S201).

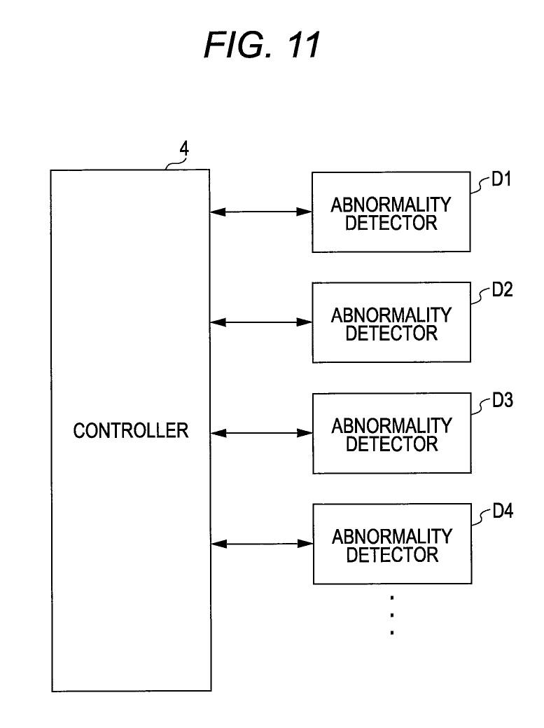

As shown in FIG. 11, the measuring section 2 has a plurality of abnormality detectors D1, D2 and the like which are connected to the controller 4. The abnormality detector D1 is a pipette crash sensor for detecting impact of the dispensing pipette provided on the sample dispensing arm 260. The pipette crash sensor generates a detection signal when the tip of the pipette strikes an obstruction while the arm is moving, and transmits the detection signal to the controller 4. The controller 4 determines that an abnormality has occurred during the measurement operation when the detection signal is received. This type of abnormality detection sensor S1 is not just provided on the sample dispensing arm 260, it is also provided on each arm mechanism, including the R1 reagent dispensing arm 261, R2 reagent dispensing arm 262, R3 reagent dispensing arm 263 and the like.

The abnormality detector D2 is a disconnection detection circuit for detecting a disconnection of the power line supplying current to the motors disposed at each processing station. The motors include motors for rotating each arm mechanism in axial directions and motors for moving the arm mechanisms in vertical directions, the arm mechanism including, for example, the sample dispensing arm 260, R1 reagent dispensing arm 261 and the like. The disconnection detection circuit has a resistor for disconnection detection disposed between the constant current power source and the motor, and outputs the value of the current flowing to the resistor to the controller 4. The controller 4 receives the output signal of the disconnection detection circuit D2, and compares the received current value to a predetermined standard value. Although a current of standard value or higher flows to the disconnection detection resistor while the motor is operating, current does not flow to the resistor connected to the motor when an interruption occurs in the power line to the motor. The controller 4 determines that a disconnection abnormality has occurred when the current value to the disconnection detection resistor is less than the standard value.

The measuring section 2 is provided with a plurality of types of abnormality detectors D3, D4 and the like in addition to the abnormality detectors D1 and D2. These abnormality detectors may, for example, detect an interruption of the power supply from an external power source, and detect leakage of liquid from cuvettes and reagent containers in the measuring section 2.

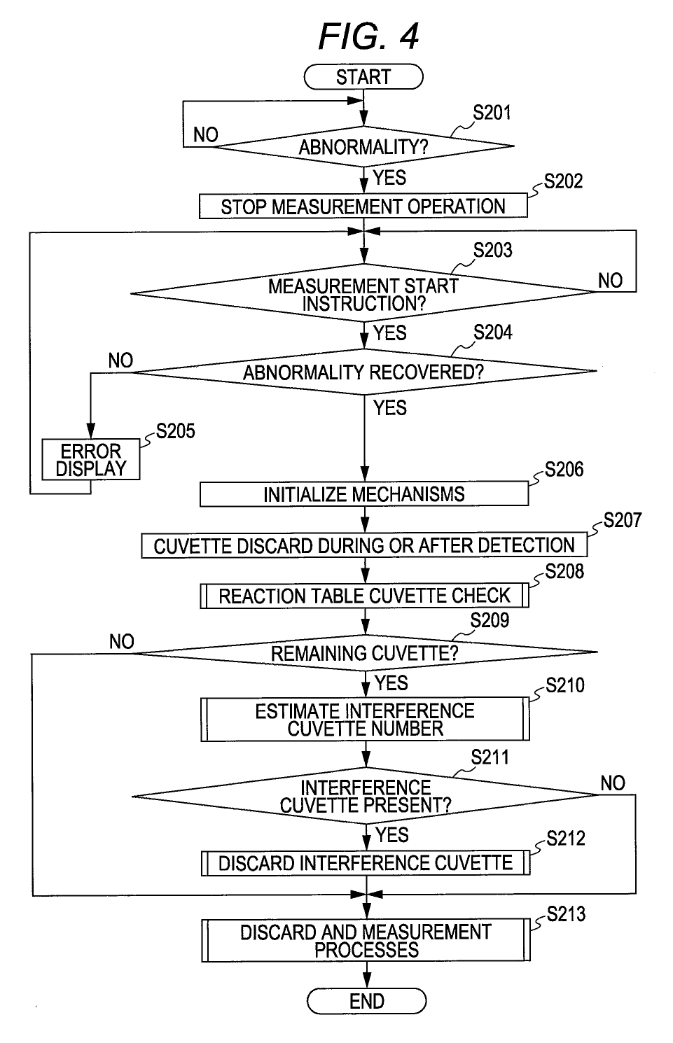

As shown in FIG. 4, the controller 4 detects an abnormality during a measurement operation via the abnormality detectors D1, D2 and the like (step S201: YES), the measurement operation is stopped (step S202). When the measurement is stopped, the operations of all processing stations are stopped, including the reaction table 200, primary BF separation section 220, and secondary BF separation section 230. Hence, the unmeasured cuvettes remain on the reaction table 200.

When an abnormality occurs and the measurement operation is stopped, the user or service person performs abnormality recovery by removing the cause of the abnormality. For example, when the measurement operation is stopped due to a pipette crash, abnormality recovery is performed by removing the obstruction that caused the pipette crash. When a disconnection is the cause of the measurement operation being stopped, abnormality recovery is performed by repairing the disconnected power line.

The controller 4 determines whether a measurement start instruction has been received from the measuring section 2 (step S203). Specifically, the controller 4 determines whether the measurement start button shown on the display/operation section 5 has been operated. When the user has not issued a measurement start instruction (step S203: NO), the controller repeats the determination. When the user has issued a measurement start instruction (step S203: YES), the controller determines whether abnormality recovery has been performed (step S204).

When abnormality recovery has not been performed (step S204: NO), the controller 4 displays, on the display/operation section 5, an error message indicating the measurement cannot restart because abnormality recovery has not been performed (step S205), and the process returns to step S203. When abnormality recovery has been performed (step S204: YES), the controller 4 initializes the mechanisms (step S206).

Initialization of the sections is an operation that returns each mechanism in the measuring section 2 to the initial position. Specifically, the controller 4 returns all mechanisms, including the sample dispensing arm 260, reagent dispensing arms 261 through 263, catchers 261a, 221, 231, 266 and the like to their initial positions.

When the initialization of each mechanism ends, the controller discards the cuvettes during detection or after detection (step S207). This process discards cuvettes when cuvettes are held by the detection section 250, catcher 266, and drain holding section 281. Specifically, when the catcher 266 holds a cuvette, the liquid in the cuvette held in the catcher 266 is removed by the drain section 282, and the cuvette is discarded to the discarding port W. When a cuvette is in the detection section 250, the catcher 266 moves the cuvette to the drain holding section 281, the liquid is removed by the drain section 282, and the cuvette is discarded in the discarding port W. When a cuvette is in the drain holding section 281, the liquid is removed by the drain section 282, the cuvette is discarded to the discarding port W.

The controller 4 performs a cuvette check for the presence of a cuvette on the reaction table 200 (step S208). The cuvette check is a process for detecting the number and positions of cuvettes remaining on the reaction table 200. Note that the cuvette check also may be executed in parallel with the process for discarding cuvettes during detection or after detection. Details of the cuvette check process are discussed later.

When the cuvette check process ends (step S208), the controller 4 determines whether a cuvette remains on the reaction table 200 (step S209). When no cuvette remains on the reaction table 200 (step S209: NO), the controller 4 skips steps S210 through S212 and the process advances to step S213.

When a cuvette remains on the reaction table 200 (step S209: YES), the controller 4 executes a process for estimating the number of interference cuvettes (step S210), and determines whether there are interference cuvettes (step S211). Note that an interference cuvette is a cuvette that may interfere with other cuvettes when measurement is restarted. The controller 4 determines that a cuvette is an interference cuvette when process for estimating the number of interference cuvettes returns a result of 1 or more, and the controller 4 determines there are no interference cuvettes when the process returns a result of 0.

When the controller 4 determines an interference cuvette is present (step S211: YES), a process is executed to discard the interference cuvette before performing measurements of the new sample (step S212). When the controller 4 determines an interference cuvette chi not present (step S211: NO), the process skips step S212 and advances to step S213. The controller 4 then starts the measurement of the new sample, and in parallel therewith, executes a process to sequentially discard the remaining cuvettes that are not interference cuvettes without performing measurements (step S213). Details will be described later.

### Cuvette Check

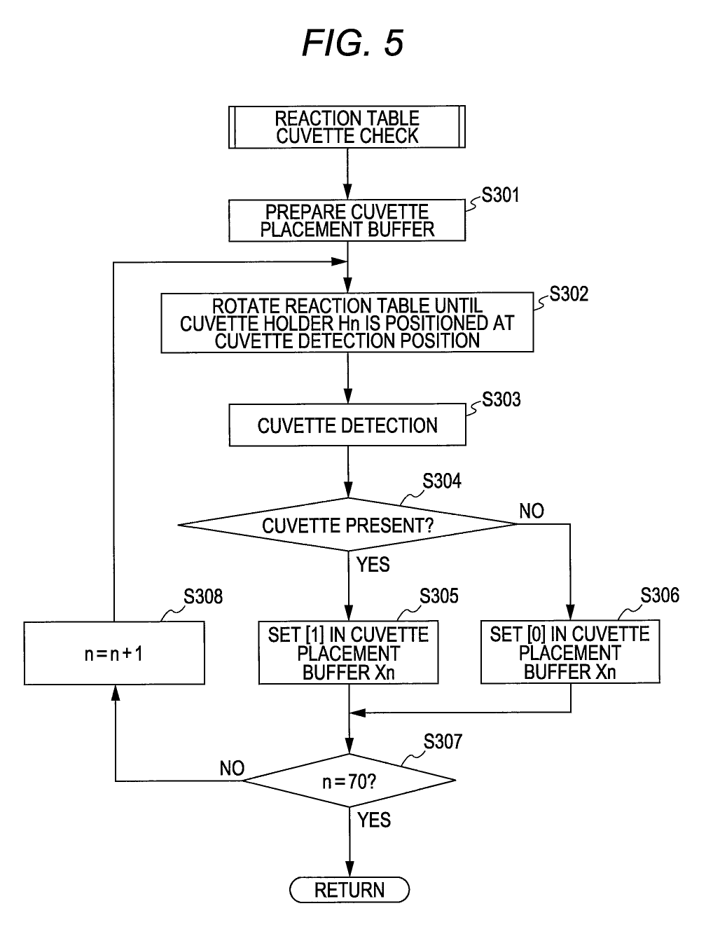

Details of the cuvette check process are described below using FIG. 5.

The controller 4 secures an empty area in RAM within the controller 4, and creates a cuvette placement buffer (step S301).

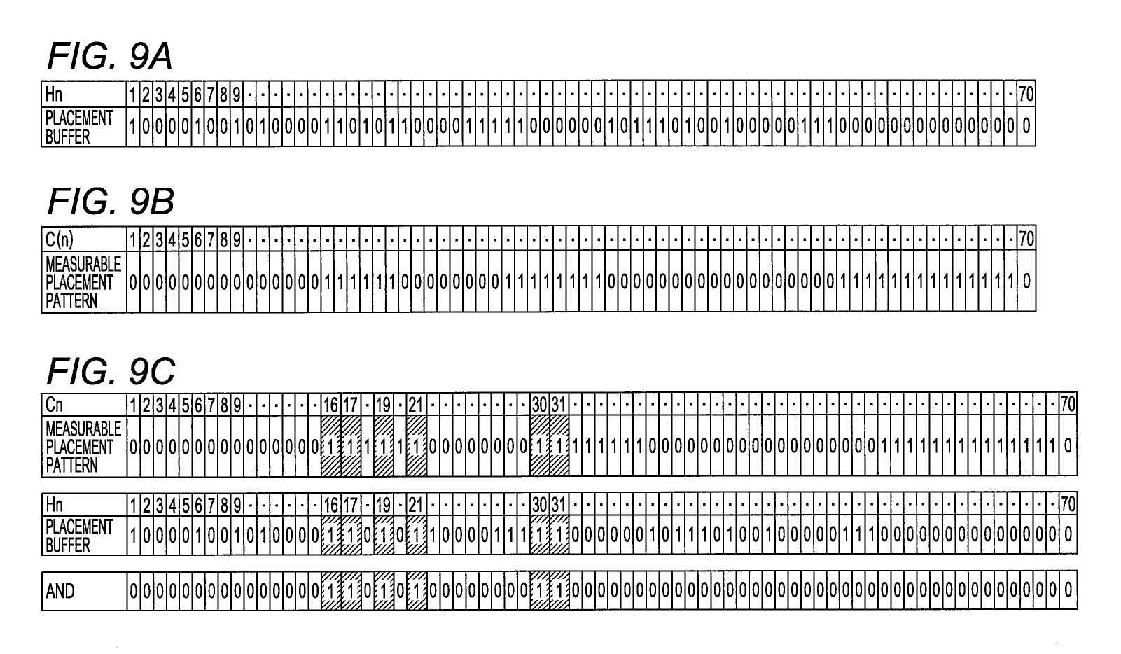

As shown in FIG. 9A, the cuvette placement buffer is a one-line table with 70 rows. Each row of the cuvette placement buffer corresponds to a cuvette holder H on the reaction table 200. For example, H1 corresponds to the first cuvette holder H, and H2 corresponds to the second cuvette holder H. The number of the cuvette holder H is conferred by allocation by the controller 4 and is not determined by the unique number of each cuvette holder H. Specifically, the controller 4 allocates a number Hn to the cuvette holder H at position Cn when the cuvette placement buffer is prepared.

A flag in the cuvette placement buffer indicates either [1: cuvette present] or [0: cuvette absent] in a cuvette holder H. At the time of step S301, each row of the buffer is blank since it is unclear whether a cuvette is present in the cuvette holder H.

The controller 4 rotates the reaction table 200 until the cuvette holder Hn is positioned at the cuvette detection position C50 (step S302). Note that n−1 is set as the initial value of n, and the cuvette holder H1 is positioned at the cuvette detection position C50 by step S302, which is executed at the start.

The controller 4 detects the presence/absence of a cuvette at the cuvette detection position C50 (step S303). Specifically, the controller 4 irradiates from the light emitting part 291 on the light receiving part 292 and acquires the amount of light in the light receiving part 292 at this time. The controller 4 determines whether a cuvette is present in the cuvette holder H (step S304). Specifically, the controller 4 determines whether the amount of light acquired in step S301 is equal to or greater than a predetermined value; when the amount of light is greater than the predetermined value the controller 4 determines a cuvette is absent (step S304: NO), whereas the controller 4 determines a cuvette is present when the amount of light is less than the predetermined value (step S304: YES).

When the controller 4 determines a cuvette is present (step S304: YES), a flag of[1] set in the nth row of the cuvette placement buffer developed in RAM (step S305). When the controller 4 determines a cuvette is absent (step S304: NO), a flag of [0] is set in the nth row of the cuvette placement buffer developed in RAM (step S306).

The controller 4 then determines whether n−70 (step S307). When n does not equal 70 (step S307: NO), the controller 4 increments n by [1] (step S308), and returns to the process of step S301. When the controller 4 determines that n=70 (step S307: YES), the subroutine ends and the process returns to the main routine. Therefore, the process of steps S302 through S306 is repeated until n=70. Hence, it can be determined whether a cuvette is present/absent when all cuvette holders H1 through H70 of the reaction table 200 are positioned at the cuvette detection position C50. A flag of [1] or [0] is thus set in all rows of the cuvette placement buffer.

### Process for Estimating Number of Interference Cuvettes

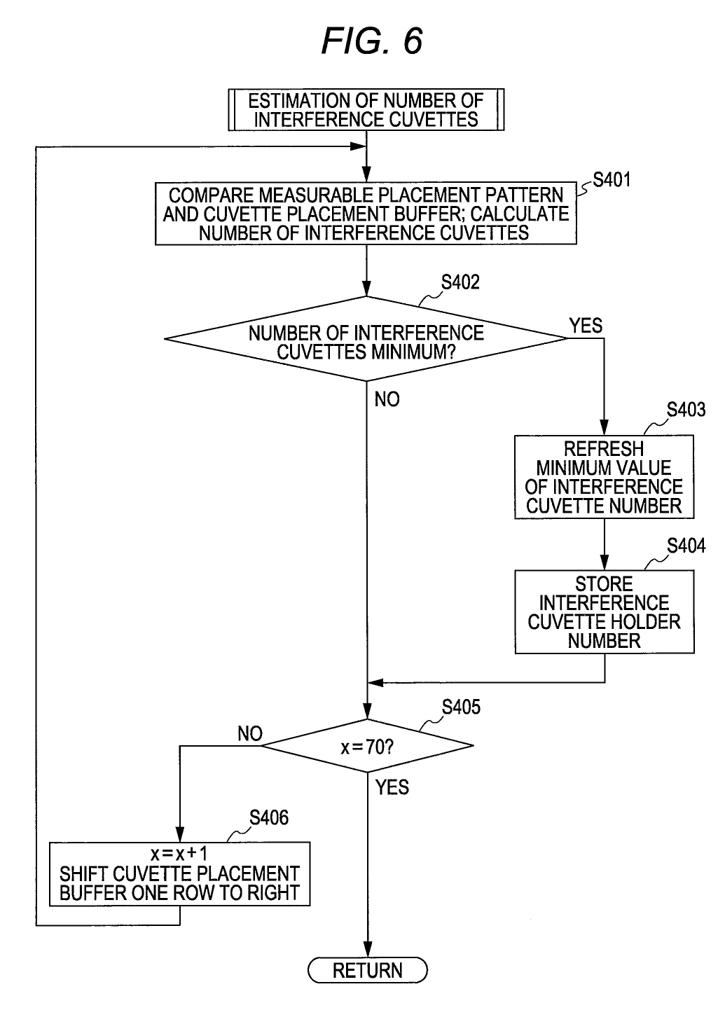

The process for estimating the number of interference cuvettes is described in detail below using FIG. 6.

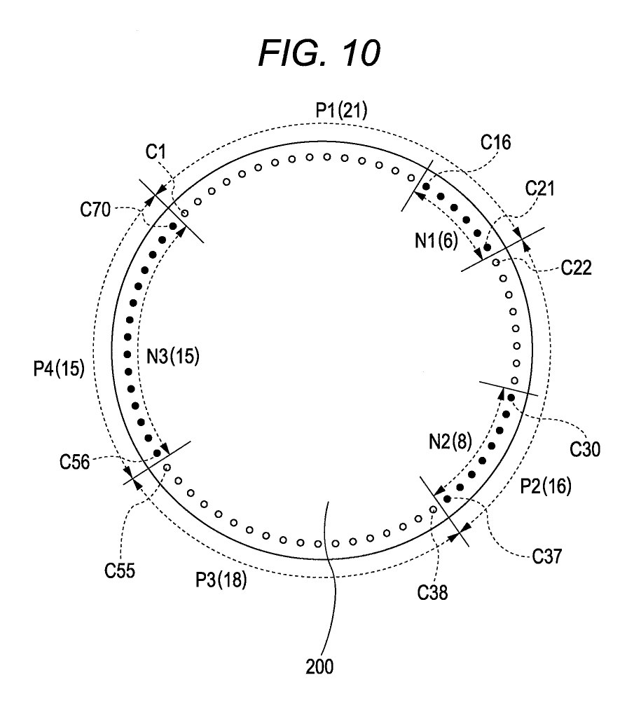

The controller 4 first compares the measurable placement pattern stored in the ROM of the controller 4 with the cuvette placement buffer (FIG. 9A) created by the cuvette check, and calculates the number of interference cuvettes (step S401). This process is described below using FIGS. 9 and 10.

The measurable placement pattern a pattern indicating cuvette placement on the reaction table 200 which allows measurement to be started without causing cuvette interference.

The seventy cuvette holders H on the reaction table 200 are broadly divided into four areas. The first is area P1 configured by twenty-one cuvette holders H from C1 to C21. The second is area P2 configured by sixteen cuvette holders H from C22 to C37. The third is area P3 configured by eighteen cuvette holders H from C38 to C55. The fourth is area P4 configured by fifteen cuvette holders H from C56 to C70.

The area P1 from C16 to C21 is an area of cuvette interference when cuvettes are held therein; in other words, area P1 becomes area (interference region) N1 in which the cuvette holders H must be empty. When a cuvette is in the interference area N1, interference between cuvettes occurs because the cuvette from the primary BF separation section 220 is returned to the reaction table 200.

The area P2 from C30 to C37 is designated interference region N2. When a cuvette is in the interference area N2, interference between cuvettes occurs because the cuvette from the secondary BF separation section 230 is returned to the reaction table 200.

The area P3 from C56 to C70 is designated interference region N3. Interference occurs between cuvettes when a cuvette is set at the transport start position C1 when a cuvette is present in the unplaceable region N3 because there is no mechanism for discarding of cuvettes downstream from C56.

Note that in FIG. 10 the cuvette holders H in the interference regions are indicated by black circles, and the cuvette holders H that do not obstruct the start of measurement even when a cuvette is present are indicated by white circles.

The measurable placement pattern shown in FIG. 10 becomes the pattern of FIG. 9B when the cuvette placement pattern is recorded as comparable data rows. In FIG. 9B, the flag [1] is set at positions corresponding to an interference region, and flag [0] is set at locations in other regions. Therefore, when the measurable placement pattern is compared to the cuvette placement buffer, it is possible to specify the number and positions of cuvettes positioned in an interference region when the reaction table 200 is at an optional angle.

The controller 4 performs an AND calculation of the measurable placement pattern of each row and the cuvette placement buffer of each row, and determines the sum of the AND calculation result of each row.

The interference region is indicated by the flag [1] in the measurable placement pattern, and the position of the cuvette is indicated by the flag [1] in the cuvette placement buffer. Therefore, a row with an AND calculation result of [1] among these rows indicates a cuvette in the row is an interference cuvette. The sum of the AND calculations indicate the number of interference cuvettes.

In the example shown in FIG. 9C, when the AND calculation is performed for each row of the measurable placement pattern and the cuvette placement buffer, the calculation result is 1 in the six rows shown in halftone, and the sum of the AND calculation results is therefore six. In this case the number of interference cuvettes is therefore six, and the interference cuvettes are located at H16, H17, H19, H21, H30, and H31.

Returning now to FIG. 6, the controller 4 determines whether the number of interference cuvettes acquired in step S401 is less than the minimum number of interference cuvettes pre-stored in memory. The minimum number of interference cuvettes for comparison in the first process is pre-set sufficiently large, for example, [70], so that the number of interference cuvettes acquired on the first process is less than the minimum number of interference cuvettes.

When the number of interference cuvettes acquired in step S401 is less than the minimum number of interference cuvettes(step S402: YES), the controller 4 stores the number of interference cuvettes in RAM as the minimum number of interference cuvettes (S403), and the number of the cuvette holder H at the head of the row in the cuvette placement buffer is also stored in RAM with the number of the cuvette holder H of the interference cuvette (S404). The controller 4 then advances the process to step S405.

When the number of interference cuvettes acquired in step S401 is equal to or greater than the minimum number of interference cuvettes (step S402: NO), the controller 4 skips steps S403 and S404, and advances the process to step S405.

The controller 4 determines whether the number of comparisons x has reached 70 (step S405). The number of comparisons x is a count of the number of times the cuvette placement buffer has been compared with the measurable placement pattern, and the initial value of x is 1. The controller 4 ends the subroutine of FIG. 6 and returns to the main routine when the number of comparisons x is 70 (step S405: YES), whereas the controller 4 advances the process to step S406 when the number of comparisons x has not reached 70 (step S405: NO).

The controller 4 increments the number of comparisons x by [1], and shifts the cuvette placement buffer one row to the right (step S406). Specifically, when the cuvette placement buffer is shifted one row to the right, the maximum unit (first row) is embedded by the value of the canceled row since minimum unit (70th row) is canceled. The controller 4 returns the process again to step S401, and the cuvette placement buffer of the row shifted to the right is compared with the measurable placement pattern, and the number of interference cuvettes is again calculated.

In this way the number of interference cuvettes can be estimated at all rotation angles of the reaction table 200 by repeating seventy times the process of comparing the measurable placement pattern each time the cuvette placement buffer is shifted 1 row to the right.

### Interference Cuvette Discarding Process

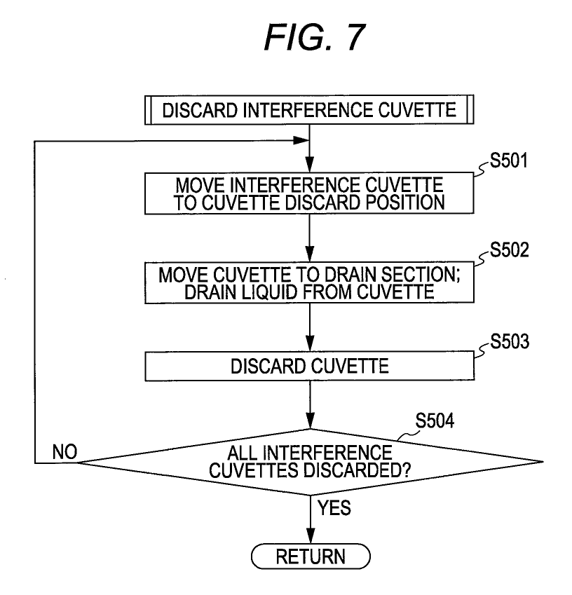

The process for discarding of interference cuvettes is described in detail below using FIG. 7.

The controller 4 rotates the reaction table 200 to position the cuvette determined to be an interference cuvette at the transport end position C56 (refer to FIG. 2) (step S501). In step S404 of FIG. 6, the number of the cuvette holder H determined to have an interference cuvette was stored in the RAM of the controller 4. The controller 4 reads out the number and rotates the reaction table 200 in the clockwise direction A1 or the counterclockwise direction to position the cuvette holder H of this number at the transport end position C56.

The controller 4 removed the cuvette positioned at the transport end position C56 via the catcher 266, sets the cuvette at the drain holding section 281, aspirates the liquid from the cuvette via the drain part 282, and discharged the liquid outside the apparatus as waste fluid (step S502).

The controller 4 grips the empty cuvette in the drain holding section 281 via the catcher 266, and discards the cuvette in the discarding port W (step S503).

The controller 4 determines whether all interference cuvettes have been discarded (step S504). When an undiscarded interference cuvette remains (step S504: NO), the controller 4 returns the process to step S501, and executes the process of steps S501 through S503 for the next interference cuvette. When all interference cuvettes are discarded (step S504: YES), the controller 4 ends the subroutine and returns to the main routine.

### Discarding and Measurement Processes

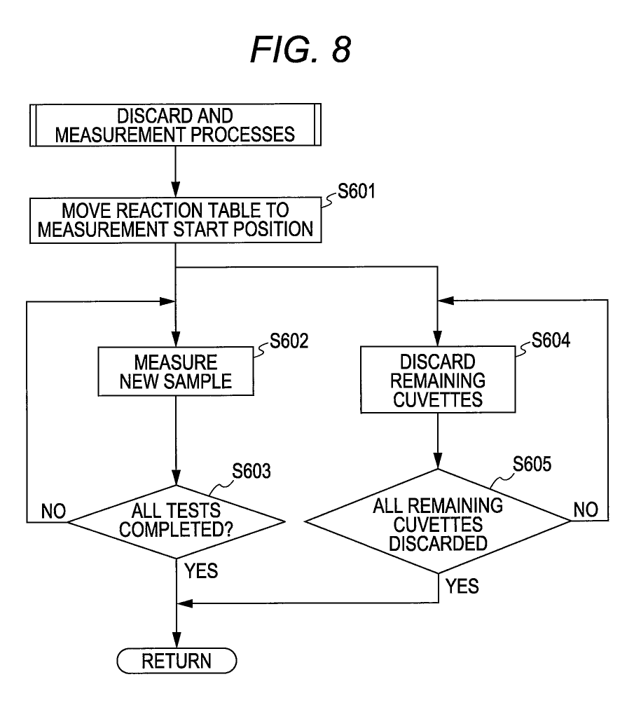

The discarding and measurement processes are described in detail below using FIG. 8.

The controller 4 first rotates the reaction table 200 to the measurement start angle (step S601). The measurement start angle is the angle of the reaction table 200 at which the cuvette holder H indicated by the number at the head of the row stored in RAM in step S404 is positioned at the transport start position C1.

The controller then starts the measurement of the new sample (step S602). The measurement flow of the new sample is shown in FIG. 3, hence, detailed description is abbreviated.

The controller 4 executes the discarding process for the remaining cuvettes in parallel with the measurement of the new sample (step S604). The remaining cuvettes are cuvettes other than the interference cuvettes among the cuvettes loaded in the reaction table 200.

The parallel processes of the measurement of the new sample and discarding of the remaining cuvettes is described in detail below.

In the measurement of the new sample, the measuring section 2 sets the cuvette containing the new sample at the transport start position C1 of the reaction table 200, and executes each process shown in FIG. 3 as the reaction table 200 is rotated a fixed angle θ each 1 turn. The remaining cuvettes set on the reaction table 200 are sequentially transported in conjunction with the rotation of the reaction table 200.

At this time the measuring section 2 executes only the moving process of the remaining cuvettes and does not perform reagent dispensing or BF separation among the processes of FIG. 3. The moving process is specifically the process described below.

S106 (move from reaction table to primary BF table);

S108 (move from primary BF table to reaction table);

S110 (move from reaction table to secondary BF table);

S112 (move from secondary BF table to reaction table);

S113 (move from reaction table to R4/R5 reagent dispensing table);

S114 (move to R4/R5 reagent dispensing table).

When the remaining cuvette reaches the transport end position C56, the catcher 266 removes the remaining cuvette from the reaction table 200, and move the cuvette to the detecting section 250 (step S115). The cuvette waits in the detecting section 250 until the next turn without measurement light. When detection is completed by the detecting section 250, the catcher 266 removes the cuvette from the detecting section 250 and moves the cuvette to the drain holding section 281, then the drain section 282 aspirates the liquid from the cuvette and discharges the liquid. The catcher 266 then removes the cuvette from the detecting section 250, moves to the drain holding section 281, and the drain part 282 aspirates and discards the liquid from the cuvette. The catcher 266 moves the cuvette to above the discarding port W, and the cuvette is discarded from the discarding port W to the discarding bag when the catcher 266 releases the grip on the cuvette (step S116).

The controller 4 determines whether all remaining cuvettes have been discarded (step S605). When all remaining cuvette are discarded (step S605: YES), the remaining cuvette discarding process ends.

The controller 4 determines whether measurement of all sample to be measured have been completed (step S603). When all measurements have been completed (step S603: YES), the new sample measurement process ends.

As described above, when the immunoanalyzer 1 of the present embodiment stops the measurement operation when an abnormality occurs and receives a measurement start instruction after the abnormality recovery process, the controller 4 sequentially executes measurement operations for cuvettes containing new samples, and in conjunction with the measurement operations of the new samples controls the cuvette moving section to sequentially move the cuvettes left unmeasured due to the stopped operation from the cuvette transporting section to the aspirating and removal section, and the cuvette discarding section.

According to this structure, measurement of new sample can be started without discarding all unmeasured cuvettes even when there are many unmeasured cuvettes remaining on the reaction table 200 due to the stopped measurement. Therefore, the time required until measurement of the new samples start is reduced.

In the immunoanalyzer of the present embodiment, a determination is made whether interference cuvettes are present, as to and when interference cuvettes are present the interference cuvettes are sequentially discarded before measurement of the new sample. This structure provided the following advantages.

To recover from an abnormality, the user performs abnormality recovery after moving the unmeasured cuvettes to a different position on the reaction table 200; in this case, the cuvette remains on the reaction table 200 in a placement pattern that is different than when the apparatus has operated normally.

The apparatus is periodically inspected and maintained for the sake of normal operation; however, the user or operator may confirm the operation by manually setting a cuvette on the reaction table 200. In this case, when there is any chance of forgetting to recover a cuvette, the cuvettes are present on the reaction table 200 in a placement pattern that differs from the pattern when the apparatus has operated normally.

In this embodiment, the determination as to the presence of interference cuvettes is made prior to starting measurement, and any present interference cuvette is discarded before starting measurement of a new sample. Therefore, measurement may be safely started without cuvette interference even when the cuvettes are present on the reaction table 200 in a placement pattern that differs from the pattern when the apparatus has operated normally.

The embodiment described above is in all respects an example and may be variously modified.

For example, although the embodiment has been described in terms of stopping the operation of all processing stations of the measuring section 2 when an abnormality is detected by abnormality detectors D1, D2 and the like, the present invention is not limited to this example. For example, as disclosed in US Patent Publication No. 2009/215183, when an abnormality is detected in a specific processing station, the processing may be stopped for cuvettes that have not yet reached that processing station, and the processing continued for those cuvettes that have already passed that processing station.

More specifically, Consider an example is described in which a motor for mixing a cuvette is disconnected in the primary BF separation section 220. In this case, processing is continued for the cuvettes already processed by the primary BF separation section 220, and more specifically, the cuvettes in processes downstream from position C22 of the reaction table 200. On the other hand, processing is stopped for the cuvettes held at the standby section 223 and four holders 224 through 227 of the primary BF separation section 220, and cuvettes held at positions C1 through C17 of the reaction table 200.

According to this structure, processing can be continued for as many cuvettes as possible, and waste of sample and reagent can be reduced.

The above embodiment is described in terms of starting the transport of the cuvette by the R1 reagent dispensing arm 261 dispensing R1 reagent to the cuvette at the R1 reagent dispensing position P2, the sample dispensing arm 260 dispensing sample to the cuvette at the sample dispensing position P3, and the catcher 261 a setting the cuvette containing the dispensed sample at the transport start position C1, it is to be understood that the present invention is not limited to this example. For example, the catcher 261a may set the empty cuvette at the transport start position C1 on the reaction table 200, then the R1 reagent dispensing arm 261 and sample dispensing arm may dispense the R1 reagent and the sample into the cuvette on the reaction table 200.

Although the detecting section 250 detects measurement object substance contained in a cuvette removed from the reaction table 200 in the above embodiment, the measurement object substance also may be detected within the cuvette while the cuvette is held on the reaction table 200.

The above embodiment has been described in terms of discarding remaining cuvettes in parallel with the measurement of the new sample by temporarily moving the remaining cuvette to the detecting section and removing liquid therefrom then discarding the cuvette identically to the flow of the measurement process of the new sample, however, the invention is not limited to this example. The remaining cuvettes may be moved directly to the drain holding section 281 without passing through the detecting section, then removing the waste liquid via the drain part 282 and discarding the liquid to the discarding port W.

Although the above embodiment described an example in which the present invention is applied to an immunoanalyzer, the invention is not limited to this example inasmuch as the present invention is also applicable to blood coagulation analyzers, and biochemical analyzers and the like. Since a blood coagulation examination has different measurement protocols depending on the measurement item, in a system devised to measure many items by holding and transporting a plurality of cuvettes on a single rotating table, the measurement items that can be measured must be limited to have the same reaction time for all cuvettes. In this regard, the present invention is well suited to an immunoanalyzer that uses a constant reaction time regardless of the measurement item, so there is no need to limit measurement items.

What is claimed is:

1. A sample analyzer comprising:
a plurality of processing stations for performing processes to obtain a measurement of a sample contained in a cuvette;
a cuvette transporting section configured to sequentially transport a plurality of cuvettes, each cuvette containing a sample, to the plurality of processing stations;
a discarding station for discarding the cuvette transported to a predetermined position by the cuvette transporting section;
an abnormality detecting section for detecting an abnormality at the plurality of processing stations; and
a controller configured for performing operations, comprising:
(a) stopping the processing of a cuvette or cuvettes when an abnormality is detected by the abnormality detecting section;
(b) when an instruction for starting the processing is received after recovery from an abnormality, transporting a cuvette or cuvettes containing a new sample or samples, held by the cuvette transporting section, to the plurality of processing stations and transporting the cuvette or cuvettes for which processing has been stopped to a predetermined position; and
(c) discarding the cuvette or cuvettes transported to the predetermined position by the discarding station.

2. The sample analyzer of claim 1, wherein
The cuvette transporting section comprises a rotating table.

3. The sample analyzer of claim 1, wherein
the plurality of processing stations comprise:
a reagent dispensing section for dispensing reagent to react with a measurement object substance contained in the sample in the cuvette transported to a first position by the cuvette transporting section; and
a detecting section for performing processing to detect the measurement object substance in the cuvette transported to a second position downstream from the first position by the cuvette transporting section.

4. The sample analyzer of claim 3, wherein
the controller is configured to control the reagent dispensing section so as to not dispense reagent to a cuvette or cuvettes containing a unmeasured sample or samples by stopping the process.

5. The sample analyzer of claim 3, wherein
the discarding station comprises:
an aspiration removal section for aspirating the liquid within a cuvette after the detecting section has performed the measurement object substance detection processing;
a cuvette discarding section for discarding the cuvette from which the liquid has been aspirated; and
a cuvette moving section capable of moving the cuvette from the detecting section to the aspiration removal section and the cuvette discarding section.

6. The sample analyzer of claim 1, further comprising:
a cuvette detecting section for detecting a cuvette in the cuvette transporting section; and
a memory unit, wherein
the controller, when a measurement start instruction is received, acquires a position information of the cuvette detected by the cuvette detecting section, and stores the position information in the memory unit.

7. The sample analyzer of claim 6, wherein
the controller, when measurement cannot be started for a cuvette in the cuvette transporting section, specifies the cuvette required to be discarded for starting measurement based on the cuvette position information stored in the memory section, and sequentially transports the specified cuvette to a predetermined position by the cuvette transporting section prior to starting measurement.

8. The sample analyzer of claim 7, wherein
the cuvette required to be discarded is a cuvette which may interfere with other cuvettes when measurement has started.

9. The sample analyzer of claim 7, wherein
the controller, after the specified cuvette has been transported to the predetermined position, moves the cuvette tansporting section to set a cuvette containing a new sample at a holding position that does not hold a cuvette in the cuvette tansporting section, and sequentially moves the cuvette containing the new sample to the plurality of processing stations.

10. The sample analyzer of claim 1, wherein
the controller, when a cuvette is present in the cuvette tansporting section, moves the cuvette tansporting section to set the cuvette containing the new sample at the holding position that does not hold a cuvette in the cuvette tansporting section, and thereafter sequentially moves the cuvette containing the new sample to the plurality of processing stations.

11. The sample analyzer of claim 1, further comprising:
a sample setting section for setting a cuvette containing a sample in the cuvette transporting section.

12. A sample analyzer comprising:
a cuvette transporting section comprising a rotating table for sequentially transporting a plurality of cuvettes, each containing a sample;
a reagent dispensing section for dispensing reagent to react with a measurement object substance contained in the sample in the cuvette transported to a first position by the cuvette transporting section;
a detecting section for performing processing to detect the measurement object substance in the cuvette transported to a second position downstream from the first position by the cuvette transporting section;
an aspiration removal section for aspirating the liquid within a cuvette after the detecting section has performed the measurement object substance detection processing;
a cuvette discarding section for discarding the cuvette from which the liquid has been aspirated;
a cuvette moving section configured to move the cuvette at the detecting section to the aspiration removal section, and move the cuvette from which the liquid has been aspirated to the cuvette discarding section;
a controller for sequentially executing measurement operations on each cuvette hold in the cuvette transporting section by controlling the operations of the reagent dispensing section, aspiration removal section, and cuvette transporting section; and
an abnormality detecting section for detecting an abnormality, wherein
the controller performs operations, comprising:
(a) stopping the measurement operation when an abnormality is detected by the abnormality detecting section during a measurement operation;
(b) when a measurement start instruction is received after the abnormality recovery process, sequentially executing measurement operations on the cuvettes containing new sample and controlling the cuvette moving section to sequentially move the cuvettes left unmeasured due to the stopped operation from the cuvette transporting section to the aspiration removal section, and the cuvette discarding section in conjunction with the measurement operation.

13. The sample analyzer of claim 12, further comprising:
a cuvette detecting section for detecting a cuvette on the rotating table; and
a memory unit, wherein
the controller, when a measurement start instruction is received, acquires a position information of the cuvette that is held on the rotating table and detected by the cuvette detecting section, and stores the position information in the memory unit.

14. The sample analyzer of claim 13, wherein
the controller, when a cuvette is present on the rotating table, rotates the rotating table to set the cuvette containing the new sample at an available position that does not hold a cuvette on the rotating table, and starts the measurement operation of the cuvette containing the new sample.

15. The sample analyzer of claim 13, wherein
the controller, when measurement cannot be started for a cuvette in the cuvette transporting section, specifies the cuvette required to be discarded for starting measurement based on the cuvette position information stored in the memory section, and controls the cuvette moving section to sequentially move the specified cuvette from the cuvette transporting section to the aspiration removal section, and the cuvette discarding section prior to starting measurement.

16. The sample analyzer of claim 15, wherein
the cuvette required to be discarded is a cuvette which may interfere with other cuvettes when measurement has started.

17. The sample analyzer of claim 15, wherein
the controller, after the specified cuvette has been discarded to the cuvette discarding section, rotates the rotating table to set the cuvette containing the new sample at an available position that does not hold a cuvette on the rotating table, and starts the measurement operation of the cuvette containing the new sample.

18. The sample analyzer of claim 12, wherein
the controller is configured to control the reagent dispensing section so as to not dispense reagent to a cuvette or cuvettes containing a unmeasured sample or samples by stopping the process.

19. The sample analyzer of claim 12, further comprising:
a sample setting section for setting a cuvette containing a sample in the cuvette transporting section.

20. The sample analyzer of claim 12, wherein
the cuvette moving section moves the cuvette transported to a third position from the cuvette transporting section to the detecting section and sequentially moves the cuvette from the detecting section to the aspiration removal section and the cuvette discarding section after detection.
